(19) United States
(12) Patent Application Publication
(10) Pub. No.: US 2022/0096765 A1
(43) Pub. Date: Mar. 31, 2022

(54) VENTILATION APPARATUS AND VENTILATION METHOD

(71) Applicant: Drägerwerk AG & Co. KGaA, Lübeck (DE)

(72) Inventors: Thomas KRÜGER, Reinfeld (DE); Birgit STENDER, Lübeck (DE)

(21) Appl. No.: 17/426,515

(22) PCT Filed: Jan. 7, 2020
(86) PCT No.: PCT/EP2020/050164

§ 371 Date: Jul. 28, 2021

(30) Foreign Application Priority Data
Jan. 29, 2019 (DE) 10 2019 000 584.8

(51) Int. Cl. A61M 16/00; G16H 20/40; G16H 40/63; G16H 50/20

(52) U.S. Cl. CPC A61M 16/024; A61M 16/0051; G16H 20/40; G16H 40/63; A61M 2205/584; A61M 2230/42; A61M 2205/3379; A61M 2205/3344; A61M 2205/505; G16H 50/20

(57) ABSTRACT

A ventilator (1), for ventilating the lungs of a patient with breathing air, includes a ventilation module (2) for generating a breathing air flow, a determination module (3) for determining a first ventilation parameter as well as a different second ventilation parameter of the ventilator, and a control module (4) for controlling the ventilator as a function of the determined first and/or second ventilation parameter. The control module is configured to reduce the first ventilation parameter automatically over an analysis period including at least one breathing cycle. A classification module (5) is configured to classify a pulmonary status of the lungs of the patient based on a change in the second ventilation parameter, which change was brought about by the automatic reduction of the first ventilation parameter. A process is further provided for ventilating the lungs of a patient with breathing air with a ventilator (1).

### CROSS REFERENCE TO RELATED APPLICATIONS

This application is a United States National Phase Application of International Application PCT/EP2020/050164, filed Jan. 7, 2020, and claims the benefit of priority under 35 U.S.C. § 119 of German Application 10 2019 000 584.8, filed Jan. 29, 2019, the entire contents of which are incorporated herein by reference.

### TECHNICAL FIELD

The present invention pertains to a ventilator for ventilating the lungs of a patient with breathing air. The ventilator has a ventilation module for generating a breathing air flow, a determination module for determining ventilation parameters of the ventilator and a control device for controlling the ventilator as a function of the ventilation parameters determined and/or of a desired ventilation specification. The present invention further pertains to a process for ventilating the lungs of a patient with breathing air by means of a ventilator.

### TECHNICAL BACKGROUND

A ventilator is defined within the framework of the present invention as a device by means of which a gas, a gas mixture, especially breathing air, an anesthetic or the like can be introduced into the lungs of the patient and can be removed from the lungs by building up a breathing pressure. An externally controlled ventilation of the lungs can thus be carried out by means of a ventilator, so that an active breathing by the patient is not necessary.

A plurality of different ventilators are known, which differ especially in the configuration and in the manner of functioning. Distinction is made, in principle, between ventilators with an open ventilation system and with a closed ventilation system. Ventilators with an open ventilation system are configured to remove used breathing air of the patient to the environment of the ventilator. Such ventilators are used especially in cases in which the ventilation medium, for example, normal breathing air, oxygen-enriched breathing air or the like, is harmless for the environment. By contrast, ventilators with a closed ventilation system have a gas outlet, via which the used breathing air can be introduced into a closed waste air duct or gas circuit.

Such ventilators are used especially in operating rooms as anesthesia devices in order thus to prevent the release of anesthetics into the environment of the patient.

A special function of some ventilators is that recruitment maneuvers are carried out to improve the pulmonary status of the lungs of the patient. Pulmonary statuses are classified essentially to three categories, namely, to collapsed, normal and overdistended. For example, the ventilation pressure or the ventilation volume can be increased in case of collapsed lungs. Collapsed regions of the lungs can expand or extend again as a result. A suitable recruitment maneuver in case of overdistended lungs is a reduction of the ventilation pressure or of the ventilation volume in order to relax the overdistended regions of the lungs. Correct determination of the pulmonary status is essential for carrying out a suitable recruitment maneuver.

It is know from the publication “Adjusting tidal volume to stress index in an open lung situation optimizes ventilation and prevents overdistension in an experimental model of lung injury and reduced chest wall compliance” by C. Ferrando et al. how a so-called “stress index” is used following lung recruitment in order to adapt the tidal volume (V_{T}). The curve describing the airway pressure is analyzed for this purpose in sections of constant volume flow.

The calculation of such a stress index is known, for example, from the publication “Airway pressure-time curve profile (stress index) detects tidal recruitment/hyperinflation in experimental acute lung injury” by S. Grasso et al. The calculation can accordingly be carried out, for example, by means of a non-linear regression analysis for a section of the phase of inhalation (inhale), in which the volume flow is maintained at a constant level.

Another approach is based on the calculation of C20/C according to the publication “Pediatr. overdistension during mechanical ventilation by using volume-pressure loops” by J. Fisher et al. The global linear lung compliance calculated from the last 20% of the lung volume curve is related here to the linear compliance calculated over the entire lung volume curve.

It is disadvantageous here that these approaches are not based on the parameter identification of a dynamic system, for example, with pressure-dependent lung compliance. The parameter estimates performed in such a procedure are not particularly robust, so that there is a high error probability.

Fisher et al. carry out a so-called “low-flow” maneuver, so that the procedure only requires here the determination of secants or tangents and quotient formation. The procedure is not applicable in both cases without a change from an ongoing pressure-controlled ventilation, because requirements are imposed on the curve of the volume flow.

Moreover, a derivation for the identification of a regional mechanical lung model on the basis of four lung compartments used as examples from impedance curves in regions of EIT images is known from the publication “On the feasibility of automated mechanical ventilation control through EIT” by H. Tregidgo et al. The lung model is described by a usual linear differential equation. The parameters “resistance” and “elastance” are estimated for different regions of the lungs on the basis of measured values of the ventilator in combination with time series of EIT images.

This procedure represents only the basis for the estimation of a distributed dynamic but linear lung model. The identification of this model describes a linearization at the working point (mean airway pressure). The compliance is independent from the time and is independent from the airway pressure or alveolar pressure in this approach. In order to detect an overdistension, it is, however, necessary to detect the nonlinear behavior of the lungs, i.e., the reduction of compliance with increasing alveolar pressure.

The publication “Bedside estimation of recruitable alveolar collapse and overdistension by electrical impedance tomography” by E. Costa et al. describes how the loss of compliance compared to the regional optimum can be determined on the basis of a titration of the PEEP (positive end-expiratory pressure) in EIT images in combination with pneumatic measured values at the mouth of the patient (volume, pressure). Overdistension is assumed in case of a reduction of compliance above the regionally different airway pressure at which the optimal regional compliance is reached and collapse is assumed below the regionally different airway pressure at which the optimal regional compliance is reached.

The drawback of this process is that a recruitment maneuver in the form of an initial elevation and then a stepwise lowering of the PEEP is necessary for this. This elevation may become problematic for the patient especially if the pulmonary status of the lungs is overdistended already before the recruitment maneuver. A further injury to the lungs cannot be ruled out in this case.

### SUMMARY

Based on this state of the art, a basic object of the present invention is to provide a ventilator as well as a process for ventilating the lungs of a patient with breathing air by means of a ventilator, which is free or at least partially free from these drawbacks. Therefore, the object of the present invention is to provide a ventilator as well as a process which guarantee a careful determination of the pulmonary status of the patient and avoid an excessive stress on the lungs.

Accordingly, the object is accomplished by a ventilator for ventilating the lungs of a patient with breathing air, which ventilator has features according to the invention, as well as by a process for ventilating the lungs of a patient with breathing air by means of a ventilator, which process has features according to the invention.

Features and details that are described in connection with the ventilator according to the present invention are, of course, also valid in connection with the process according to the present invention and vice versa, so that reference is and can always mutually be made to the individual aspects of the present invention concerning the disclosure.

According to a first aspect of the present invention, the object is accomplished by a ventilator for ventilating the lungs of a patient with breathing air. The ventilator has a ventilation module for generating a breathing air flow, a determination module for determining a first ventilation parameter as well as a second ventilation parameter of the ventilator, which parameter is different from the first ventilation parameter, and a control module for controlling the ventilator as a function of the determined first ventilation parameter and/or of the determined second ventilation parameter. The control module is configured according to the present invention to automatically reduce the first ventilation parameter over an analysis period comprising at least one breathing cycle.

Further, the ventilator has a classification module, said classification module being configured to classify the pulmonary status of the lungs of the patient on the basis of a change in the second ventilation parameter, which change was brought about by the automatic reduction of the first ventilation parameter.

The ventilator preferably has an inhalation tube port for the fluid-communicating coupling of the ventilator to a patient inhalation port of the patient. The ventilator preferably has an inhalation valve arranged in a fluid-communicating manner with the inhalation tube port for controlling the flow of the breathing air.

The inhalation valve is preferably arranged in the interior of the ventilator in front of the inhalation tube port in the direction of flow of the breathing air. Further, the ventilator preferably has an exhalation tube port for the fluid-communicating coupling of the ventilator to a patient exhalation port of the patient. The ventilator preferably has an exhalation valve arranged in a fluid-communicating manner with the exhalation tube port for controlling the flow of breathing air.

The exhalation valve is preferably arranged in the interior of the ventilator behind the exhalation tube port in the direction of flow of the breathing air. The ventilator may have according to the present invention an open and/or closed breathing circuit. In case of an open breathing circuit, the ventilator is configured to remove used breathing air to an environment of the ventilator. In case of a closed breathing circuit, the ventilator is configured to feed used breathing air to a breathing air circuit and thus to avoid discharge of the used breathing air into the environment of the ventilator.

The ventilation module is configured to generate the breathing air flow and it can be controlled by the control module. The determination module may be integrated, for example, completely or at least partially into the ventilator module. As an alternative or in addition, provisions may be made within the framework of the present invention for the control module to be fully or at least partially integrated into the ventilation module.

The first ventilation parameter and the second ventilation parameter can be determined by means of the determination module. Ventilation parameters are, for example, a ventilation pressure and a ventilation volume. To determine the ventilation parameters, the determination module preferably has a plurality of different sensors, especially at least one pressure sensor as well as at least one volume flow sensor. It is possible to arrange, for example, a first pressure sensor at the inhalation valve or at the inhalation tube port and a second pressure sensor at the exhalation valve or at the exhalation tube port.

By balancing on the basis of the measurement results of the first pressure sensor and of the second sensor and possibly by taking into account fluidic properties of the breathing tubes and possibly other patient-side ventilation devices in the breathing air flow between the inhalation tube port and the exhalation tube port, for example, flexibilities, material properties and surface properties or the like, it is thus possible to determine the ventilation pressure present at the patient by means of the determination module. Further, the determination module is configured for the continuous and/or intermittent determination of the ventilation parameters. The determination module is configured to determine at least the first ventilation parameter and the second ventilation parameter prior to the automatic reduction of the first ventilation parameter as well as after the automatic reduction of the first ventilation parameter.

The control device is configured to control the ventilation module, especially on the basis of the ventilation parameters determined by the determination module. Taking the determined ventilation parameters into account has the advantage that the ventilation module can be controlled especially precisely in order to be able to generate as accurate ventilation pressures as well as ventilation volumes as possible at the patient. In addition, the control device is configured automatically to initiate a process for determining the pulmonary status of the lungs. The control device is set up for this purpose to automatically reduce the first ventilation parameter by a reduction factor over an analysis period comprising at least one breathing cycle.

A breathing cycle comprises an exhalation and an inhalation. The analysis period preferably has a plurality of breathing cycles, especially between three and ten, and especially preferably five breathing cycles. The control device is preferably configured to carry out the reduction of the first ventilation parameter abruptly or gradually. The ventilator preferably has an input module, via which the reduction factor for reducing the first ventilation parameter can be set. It is possible in this manner, for example, to set a lower reduction factor for a patient with a history of collapsed lungs than for a patient with a history of overdistended lungs in order to avoid an unintended collapse of the lungs as a consequence of the reduction of the first ventilation parameter. A maximum reduction factor preferably equals 0.4, so that a robust classification can be carried out and continuous ventilation continues to be ensured.

The classification module is configured to classify the pulmonary status of the lungs of the patient by taking into account the first ventilation parameter and the second ventilation parameter, which were determined prior to the automatic reduction of the first ventilation parameter, as well as the first ventilation parameter and the second ventilation parameter determined after the automatic reduction of the first ventilation parameter. For example, a ventilation pressure and a ventilation volume may be used as ventilation parameters. A ventilation pressure or driving pressure (dP) is defined within the framework of the present invention as a measured pressure difference between an end-inspiratory plateau of the airway pressure (Pₚₗₐₜ) and a positive end-expiratory airway pressure (PEEP). The ventilation volume will also be called tidal volume (V_{T}) and it denotes the value of a breathing air volume introduced during a complete breathing cycle. A change in dP may be brought about in pressure-controlled ventilation modes by changing the preset set points for the expiratory pressure level (PEEPₛₑₜ) or for the inspiratory pressure level (P_{insp.set}). In volume-controlled ventilation modes, a change in dP can be brought about by PEEPₛₑₜ or by changing the preset set point for V_{T} (V_{T.set}).

Three different scenarios are preferred according to the present invention for the automatic reduction of the first ventilation parameter. According to a first scenario, a ventilation pressure is used as the first ventilation parameter. At constant PEEPₛₑₜ, the P_{insp.set} is reduced and the ventilation volume is monitored as the second ventilation parameter. If the ratio of ventilation volume to ventilation pressure (V_{T}/dP) increases, overdistension is present. If the ratio of the ventilation volume to the ventilation pressure decreases, a collapse is present.

According to a second scenario, a ventilation pressure is likewise used and reduced as the first ventilation parameter. In this case, P_{insp.set} is raised at constant P_{insp.set} and it is monitored as a second ventilation parameter. If the ratio of the ventilation volume to the ventilation pressure rises, a collapse is present. If the ratio of ventilation volume to ventilation pressure decreases, overdistension is present.

According to a third scenario, a ventilation volume is used as the first ventilation parameter. The ventilation volume is reduced and the ventilation pressure is monitored as the second ventilation parameter. If the ratio of ventilation volume to ventilation pressure rises, overdistension is present. If the ratio of ventilation volume to ventilation pressure drops, a collapse is present. A constant ratio of ventilation volume to ventilation pressure means for all three scenarios that the pulmonary status is normal.

A ventilator according to the present invention has the advantage over conventional ventilators that an automatic classification of the pulmonary status of the lungs of the patient can be carried out with simple means as well as in a cost-effective manner. Moreover, the ventilator according to the present invention is configured to be gentle on the lungs of the patient and thus to avoid an exacerbation of the pulmonary status. Finally, the automatic classification of the pulmonary status offers the advantage that a suitable recruitment maneuver for improving the pulmonary status can easily be identified or even carried out automatically. Optimization of the ventilation of the patient, which can especially be carried out cyclically, can thus be achieved by means of the ventilator according to the present invention. It is preferred according to the present invention that the control module is configured to carry out a recruitment maneuver to improve the pulmonary status corresponding to a classification of the pulmonary status of the lungs of the patient, which was carried out by the classification module.

Improving or an improvement is defined within the framework of the present invention especially as a measure by means of which the pulmonary status is changed in the direction of a pulmonary status that can be classified as normal. Especially an automatic adaptation of the first ventilation parameter and/or of the second ventilation parameter are taken into account for this. Suitable recruitment maneuvers may be stored, for example, in the form of a decision matrix or the like in a memory module of the ventilator.

The control device is thus able to select a suitable recruitment maneuver if the pulmonary status is known.

In case the pulmonary status is classified as collapsed, a recruitment maneuver, which counteracts collapsed lungs, can thus be carried out automatically.

For example, increasing the mean airway pressure, especially by increasing the PEEPₛₑₜ at constant dP or V_{T}, are considered for this purpose.

In case the pulmonary status is classified as overextended, a lowering of the mean airway pressure can be carried out automatically, which counteracts overextended lungs. For example, a reduction of PEEPₛₑₜ at constant dP or V_{T} is considered for this purpose. Such a control module has the advantage that the therapy of the lungs can be optimized by means of the automation. Problems of the lungs can be rapidly identified and eliminated without an intervention by an operating person being necessary for this.

Further, it is preferred that the classification module is configured to classify the pulmonary status of the lungs of the patient quantitatively as collapsed, overextended or normal. These pulmonary statuses are well suited for use as the basis for the selection of a recruitment maneuver to improve the pulmonary status. It is preferred in this connection that the ventilator is configured for the iterative automatic performance of recruitment maneuvers, so that a pulmonary status can be improved in small steps and an unintended overextension of the lungs by an unnecessary or unsuitable recruitment maneuver can be avoided.

The classification module of the ventilator is preferably configured here such as to carry out a classification of the pulmonary status automatically during ventilation maneuvers or during the ventilation maneuver, wherein the ventilator is preferably configured to carry out a suitable recruitment maneuver by means of the control device on the basis of this classification or to propose a suitable recruitment maneuver for improving the pulmonary status to an operating person.

The classification module is preferably configured quantitatively to classify the pulmonary status of the lungs of the patient. A quantitative classification of the pulmonary status is defined within the framework of the present invention especially as an indication of a degree of collapse as well as of a degree of overdistension of the lungs. The quantitative classification has the advantage that the intensity of a suitable recruitment maneuver can be derived from this. If a relatively high degree of deviation from the normal state is determined, a recruitment maneuver with a more distinct increase in the mean airway pressure can thus be identified than in case of a relatively low degree of deviation. This leads to the advantage that the number of recruitment maneuvers needed to achieve a normal pulmonary status can be markedly reduced. A time period between the identification of a pulmonary status and the establishment of the normal pulmonary status can also be reduced in this manner in an advantageous manner as well as with cost-effective steps.

According to a preferred variant of the present invention, provisions may be made in a ventilator for the ventilator to have an alarm device, said alarm device being configured to output an alarm when the quantitatively classified pulmonary status drops below a collapse limit value or exceeds an overdistension limit value. A collapse limit value is defined within the framework of the present invention as a degree of collapse of the lungs at which a recruitment maneuver should be carried out urgently to improve the pulmonary status in order to counteract an exacerbation of the health status of the patient. Falling below the collapse limit value means here that the degree of collapse of the lungs continues to increase. An overdistension limit value is defined within the framework of the present invention as a degree of overdistension of the lungs at which a recruitment maneuver should be carried out urgently to improve the pulmonary status in order to counteract an exacerbation of the health status of the patient. Exceeding the overdistension limit value means here that the degree of overdistension of the lungs continues to increase. An alarm device has the advantage that a critical pulmonary status of the lungs of the patient can be indicated to a person operating the ventilator with simple means as well as in a cost-effective manner, so that the operating person can carry out suitable countermeasures, for example, recruitment maneuvers, the administration of drugs or the like.

The control module is preferably configured automatically to reduce a ventilation volume and/or a ventilation pressure as a first ventilation parameter. The ventilation volume and the ventilation pressure are two essential ventilation parameters, which are proportional to one another in lungs with a normal pulmonary status within certain ventilation limit values. The pulmonary status can be determined from deviations of this proportionality by means of the classification module with simple means.

It is preferred that the control module is configured to reduce the first ventilation parameter stepwise over an analysis period comprising a plurality of breathing cycles. A stepwise reduction is defined within the framework of the present invention especially as an abrupt reduction of the first ventilation parameter, for example, a reduction by 10% or by 5% per reduction step.

Further, the control module is preferably configured to carry out equal reduction steps in the process. Further, the control device is preferably configured to continuously reduce the value of the reduction steps. A first reduction step is thus greater than a second reduction step and the second reduction step is greater than the next reduction step.

The control module is preferably configured to carry out one reduction step per breathing cycle. This has the advantage that an especially rapid as well as robust classification can be carried out, and an excessive stress on the lungs can be avoided.

According to a preferred configuration of the present invention, the ventilator has a display device, wherein said display device is configured to display the pulmonary status of the patient and/or to display a recruitment maneuver recommended on the basis of the pulmonary status. The display device is preferably configured as a touchscreen. Further, the display device is preferably configured as a device separate from the basic device of the ventilator and can be coupled to the basic device by means of a data cable and/or of a power cable and/or via a wireless data link. A display device has the advantage that the classified pulmonary status can be easily displayed for the person operating the ventilator. The display device is preferably configured to display the pulmonary status with the use of a color code, especially a color spectrum. The color codes can preferably be displayed in the background of the display device. An operating person can recognize in this manner by briefly looking at the display device already on the basis of the color of the background whether the pulmonary status is normal, overdistended or collapsed. A degree of the overdistension or of collapse can be displayed by the color spectrum. The display of the recommended recruitment maneuver has the advantage that a recommended action advantageous for the patient can be displayed in this manner for the operating person, so that a rapid as well as correct intervention by the operating person is improved.

The classification module is preferably configured to estimate a linear lung model of the lungs of the patient on the basis of first ventilation parameter and second ventilation parameter, which were determined prior to the automatic reduction of the first ventilation parameter, wherein the classification module is further configured to classify the pulmonary status of the lungs on the basis of the estimated lung model and on the basis of the second ventilation parameter determined after the automatic reduction of the first ventilation parameter. The classification module is preferably configured to estimate the linear lung model on the basis of measured value curves of all breathing cycles and/or of EIT data of the lungs. The linear lung model can preferably be described by means of the following differential equation:

$$\frac{{dp}_{alv}}{dt} = \frac{{p}_{aw} - {p}_{alv}}{R \times C} \qquad Formula 1$$

The state variable pₐₗᵥ designates here the pressure as a function of the compliance of the lungs. The airway pressure is designated by p_{aw}. R denotes the resistance of the lungs and C the compliance of the lungs. This linear lung model is typically valid approximately as an approximation only in a certain range of pₐₗᵥ and of p_{aw}. To determine whether a collapse or an overdistension of the lungs is present, the classification module is configured to compare ventilation volume flows determined before and after the change in the first ventilation parameter to corresponding simulated curves of the ventilation volume flow as well as of the ventilation volume on the basis of the linear lung model. A linear lung model has the advantage that additional qualitative information on the pulmonary status of the patient can be generated hereby, so that the reliability of the ventilator is improved with simple means as well as in a cost-effective manner.

It is preferred according to the present invention that the ventilator has an EIT module for detecting a pulmonary status of the lungs or at least of a part of the lungs of the patient, and the classification module is configured to take into account a change in distension and/or compliance, which was brought about after the automatic reduction of the first ventilation parameter and was detected by the EIT module during the classification of the pulmonary status. The EIT module is preferably configured to analyze the entire lung and/or individual regions of the lungs. The EIT module is configured to determine the resistance and/or the compliance of the lungs or of individual regions of the lungs and to transmit them as EIT data to the classification module. The classification module is configured to determine the pulmonary status on the basis of the change in the second ventilation parameter and in the EIT data of the EIT module, the pulmonary status, especially regional pulmonary statuses. An additional EIT module has the advantage that regional parameters of the lungs can be determined with simple means as well as in a cost-effective manner.

It is thus possible, for example, to detect local collapses and/or local overdistensions. Moreover, an automatic selection of a suitable recruitment maneuver is possible on this basis for the therapy of the lungs of the patient by the ventilator.

The control device is preferably configured to reduce the first ventilation parameter automatically by between 20% and 60%, preferably by between 30% and 50% and especially preferably by 40%. Such a reduction of the first ventilation parameter has the advantage that a robust determination of the pulmonary status can be provided in case of a comparatively slight exacerbation of the patient. An exacerbation of the health status of the patient is thus accepted to a necessary extent only in order to guarantee a reliable and robust diagnosis of the pulmonary status.

According to a second aspect of the present invention, the object is accomplished by a process for ventilating the lungs of a patient with breathing air by means of a ventilator. The process has the following process steps:
generation of a breathing air flow by means of a ventilation module of the ventilator,
determination of a first ventilation parameter and of a second ventilation parameter different from the first ventilation parameter by means of a determination module of the ventilator,
automatic reduction of the first ventilation parameter over an analysis period comprising at least one breathing cycle by means of a control device of the ventilator,
determination of a change in the second ventilation parameter brought about by the automatic reduction of the first ventilation parameter by means of the determination module, and
classification of a pulmonary status of the lungs of the patient on the basis of the change in the second ventilation parameter brought about by the automatic reduction of the first ventilation parameter by means of a classification module of the ventilator.

The ventilation module is preferably controlled by means of the control device on the basis of the first ventilation parameter and/or second ventilation parameter determined by the determination module. A breathing air flow with a predefined first ventilation parameter and with a predefined second ventilation parameter can be generated in this manner by means of the ventilation module for ventilating the lungs of the patient.

The first ventilation parameter as well as the second ventilation parameter are determined by the determination module preferably continuously or at least at regular intervals in order to guarantee a continuous ventilation of the lungs with constant ventilation parameters. Moreover, changes in the pulmonary status, for example, an abrupt collapse of the lungs, can be determined in this manner. The determination of the first ventilation parameter and of the second ventilation parameter is carried out both prior to the automatic reduction of the first ventilation parameter and thereafter.

The ventilation module is actuated by means of the control device such that the first ventilation parameter is reduced over the analysis period. The reduction is preferably carried out by between 20% and 60%, preferably by between 30% and 50% and especially preferably by 40%.

Second ventilation parameters having undergone such a change, by means of which a reliable and robust classification of the pulmonary status of the lungs is guaranteed, can be determined on the basis of such a reduction of the first ventilation parameter. In addition, the lungs of the patient are stressed only slightly during such a reduction of the first ventilation parameter.

The pulmonary status of the lungs is classified by means of the classification module of the ventilator on the basis of the change in the second ventilation parameter, which change was brought about by the automatic reduction of the first ventilation parameter. This can be carried out, for example, by comparing the quotients of the first ventilation and the second ventilation parameter prior to the automatic reduction of the first ventilation parameter and thereafter.

All the aspects that were already described in connection with a ventilator according to the first aspect of the present invention are obtained in the process according to the present invention. Accordingly, the process according to the present invention for ventilating the lungs of a patient with breathing air by means of a ventilator has the advantage over conventional processes that an automatic classification of the pulmonary status of the lungs of the patient can be carried out with simple means as well as in a cost-effective manner.

Moreover, the lungs of the patient are protected during a recruitment maneuver when the process according to the present invention is carried out compared to conventional processes, during which a classification of the pulmonary status is carried out during a recruitment maneuver, because no recruitment process but only a reduction of the first ventilation parameter is carried out according to the present invention. The risk of causing an exacerbation of the pulmonary status is considerably reduced in this manner. Finally, the automatic classification of the pulmonary status carried out by means of the process according to the present invention has the advantage that a suitable recruitment maneuver for improving the pulmonary status can easily be determined or even carried out automatically. An optimization of the ventilation of the patient, which can especially be carried out cyclically, can thus be accomplished by means of the process according to the present invention.

Provisions may be made according to the present invention in a process for using a breathing pressure as the first ventilation parameter and a ventilation volume as the second ventilation parameter. The ventilation volume and the ventilation pressure are two essential ventilation parameters, which are proportional to one another in lungs with a normal pulmonary status within certain ventilation limit values. The pulmonary status can be determined from deviations of this proportionality by means of the classification module with simple means.

The classified pulmonary status of the lungs of the patient and/or a recruitment maneuver suitable for improving the pulmonary status of the lungs are preferably displayed by means of a display device of the ventilator. As an alternative or in addition, a recruitment maneuver suitable for improving the pulmonary status of the lungs is carried out by means of the control device. Only the classified pulmonary status is displayed in the simplest case. A person operating the ventilator can identify and initiate a suitable recruitment maneuver on the basis of this information as well as his professional competency. By predefining a suitable recruitment maneuver, the operating person is relieved of the burden of identifying the suitable recruitment maneuver. Only the initiation of the recruitment maneuver is to be carried out by the operating person. In case of a fully automatic ventilator, the suitable recruitment maneuver identified by the ventilator is carried out automatically. During the recruitment maneuver, the control device transmits corresponding instructions, for example, for a reduction or for an increase in the ventilation pressure or of the ventilation volume, to the ventilation module. Intervention by the operating person is not necessary any longer in this case. The person operating the ventilator is additionally relieved hereby.

The process according to the present invention is preferably carried out by means of a ventilator according to the present invention. It is accordingly preferred that the ventilator according to the present invention is configured for carrying out the process according to the present invention. A classification of the pulmonary status of the lungs of the patient, which is gentle on the lungs, is ensured in this manner.

Further steps improving the present invention appear from the following description of some exemplary embodiments of the present invention, which are shown in the figures. Features and/or advantages, including design details and arrangements in space, which appear from the claims, from the description or from the drawings, may be important for the present invention both in themselves and in the different combinations. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a preferred embodiment of a ventilator according to the present invention;

FIG. 2 is a time diagram showing a response of collapsed lungs to a first reduction of the ventilation pressure;

FIG. 3 is a time diagram showing a response of overextended lungs to the first reduction of the ventilation pressure;

FIG. 4 is a time diagram showing a response of collapsed lungs to a second reduction of the ventilation pressure;

FIG. 5 is a time diagram showing a response of overdistended lungs to the second reduction of the ventilation pressure;

FIG. 6 shows time diagrams of pressures and volumes of collapsed lungs compared to a first linear lung model;

FIG. 7 shows time diagrams of pressures and volumes of overdistended lungs compared to a second linear lung model; and

FIG. 8 is a flow chart of a preferred embodiment of the process according to the present invention.

### DESCRIPTION OF PREFERRED EMBODIMENTS

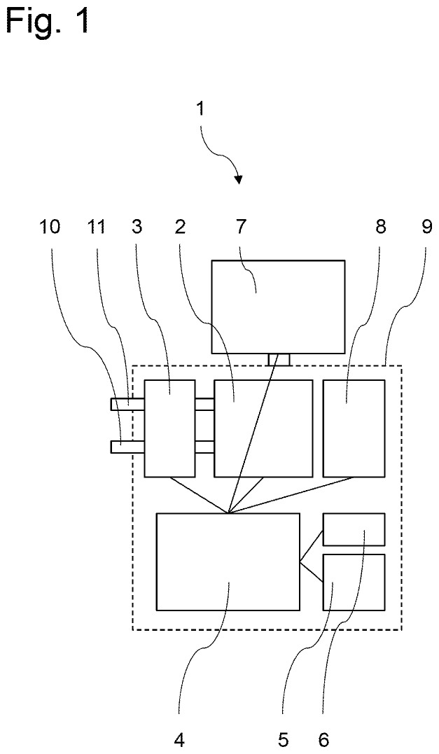

Referring to the drawings, elements having the same function and mode of operation are provided with the same reference numbers in FIGS. 1 through 8.

The preferred embodiment of a ventilator 1 according to the present invention, which is schematically shown in FIG. 1, has a ventilation module 2 for generating a breathing air flow for ventilating the lungs of a patient. The ventilation module 2 is coupled to a patient inhalation interface 10 and to a patient exhalation interface 11 in a fluid-communicating manner. Moreover, the ventilator 1 preferably has an air inlet and/or oxygen inlet and/or an anesthetic gas inlet and/or a breathing air outlet, which are not shown, and which are coupled to the patient inhalation interface 10, to the patient exhalation interface 11 and to the ventilation module 2 in a fluid-communicating manner and can be coupled via a breathing air tube for ventilating the lungs of the patient in a fluid-communicating manner. The patient inhalation interface 10 can be coupled via a breathing air tube, not shown, in order to ventilate the patient via the breathing air tube. The patient exhalation interface 11 can be coupled to the breathing air tube in order to remove breathing air from the patient to the ventilator 1. In addition, the course of the exhalation of the patient can be better controlled hereby, especially by setting or adjusting the PEEP. The patient's lungs can be prevented from collapsing in this manner.

In the preferred embodiment of the present invention that is shown in FIG. 1, a determination module 3 is coupled to the patient inhalation interface 10 and to the patient exhalation interface 11 such that air pressures as well as air volume flows in the patient inhalation interface 10 as well as in the patient exhalation interface 11 can be determined by means of the determination device 3. In addition, provisions may be made according to the present invention for the determination device 3 to have additional sensors, for example, a temperature sensor, a humidity sensor or the like in order to determine additional parameters of the air flows within and outside the ventilator. The determination device 3 is thus configured to determine the first ventilation parameter, especially a ventilation volume, and the second ventilation parameter, especially a ventilation pressure.

The ventilator 1 has a control module 4 for controlling the ventilator 1 as a function of the first ventilation parameter determined by the determination module 3 and/or of the determined second ventilation parameter. The control module 4 is thus configured to control the ventilation module 2, especially automatically to reduce the first ventilation parameter over an analysis period comprising at least one breathing cycle. Further, the ventilator 1 has a classification module 5, which is configured to classify a pulmonary status of the lungs of the patient on the basis of a change in the second ventilation parameter, which change is brought about by the automatic reduction of the first ventilation parameter. The ventilator 1 has an optional alarm device 6 in this preferred embodiment. The alarm device 6 is configured to output an alarm, especially an optical and/or acoustic alarm, when the quantitatively classified pulmonary status falls below a collapse limit value or exceeds an overdistension limit value.

Moreover, the ventilator 1 has an EIT module 8 for determining a pulmonary status of the lungs or at least of a part of the lungs of the patient. The ventilation module 2, the determination module 3, the control module 4, the classification module 5, the alarm device 6 and the EIT module 8 are arranged within a housing 9 of the ventilator 1. Provisions may be made for one or more of these components, for example, the alarm device 6 or an ET module 8, to be arranged completely or at least partially outside the housing 9. The ventilator 1 preferably has an electrode interface, not shown, for coupling patient electrodes to the EIT module.

Furthermore, the ventilator 1 has a display device 7 for displaying ventilation parameters. The display device 7 is preferably configured to display actuation information for the improved actuation of the ventilator 1. Provisions may be made according to the present invention for the display device 7 to be configured as a touchscreen. The alarm device 6 may also be integrated at least partly in the display device 7, so that the display device is configured for displaying and/or acoustically outputting alarms. The display device 7 is arranged in this exemplary embodiment outside the housing 9 and is held at same adjustably, for example, rotatably about a vertical axis and/or pivotably about a horizontal axis. Provisions may also be made for the display device 7 to be arranged completely or at least partially within the housing 9, for example, behind a window. Provisions may likewise be made according to the present invention for the display device 7 to be configured such that it is detachable from the housing 9.

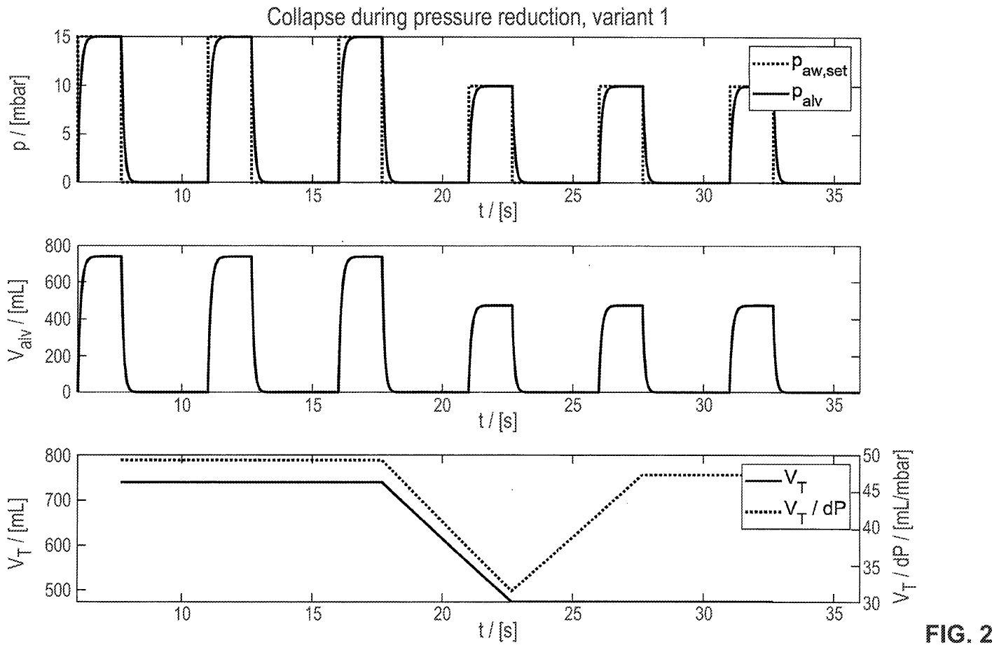

A response of collapsed lungs to a first ventilation pressure reduction is shown schematically in a diagram in a schematic time diagram in FIG. 2. The first four breathing cycles take place with non-adapted ventilation parameters. The ventilation pressure dP is reduced by the fifth breathing cycle by reducing P_{insp.set} at constant PEEPₛₑₜ. This brings about a reduction of the ventilation volume. The quotient of the ventilation volume (V_{T}) and ventilation pressure (dP) (V_{T}/dP) drops in this case. The classification module 5 can recognize from this that a collapse of the lungs is present.

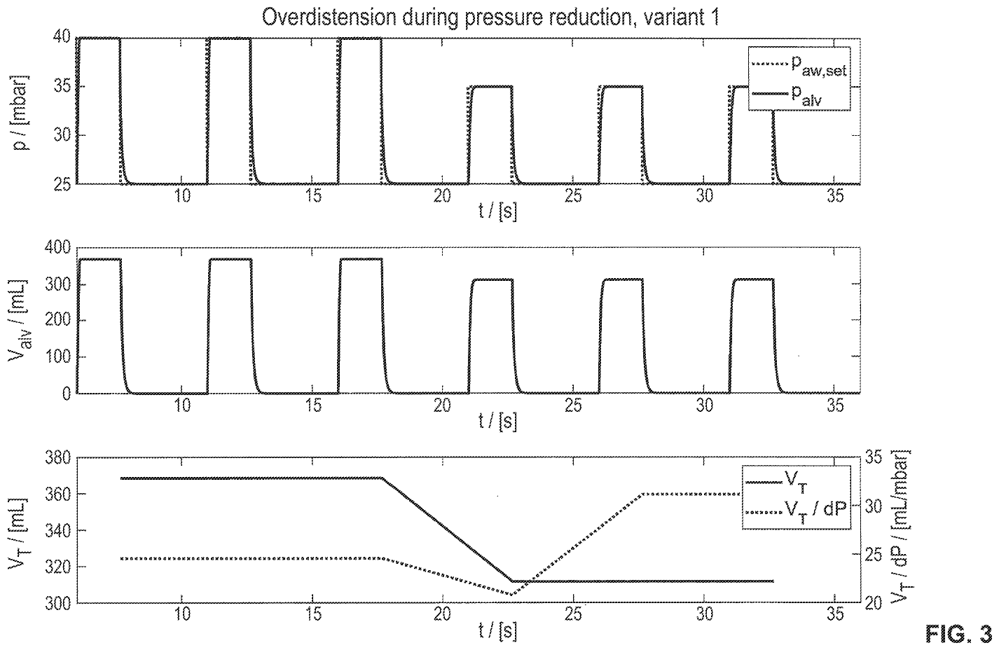

FIG. 3 schematically shows in a time diagram a response of overdistended lungs to the first ventilation pressure reduction. The first four breathing cycles take place with non-adapted ventilation parameters. The ventilation pressure is reduced by the fifth breathing cycle by reducing P_{insp.set} at constant PEEPₛₑₜ. This brings about a reduction of the ventilation volume. The quotient of the ventilation volume (V_{T}) and the ventilation pressure (dP) (V_{T}/dP) increases in this case. The classification module 5 can recognize from this that an overdistension of the lungs is present.

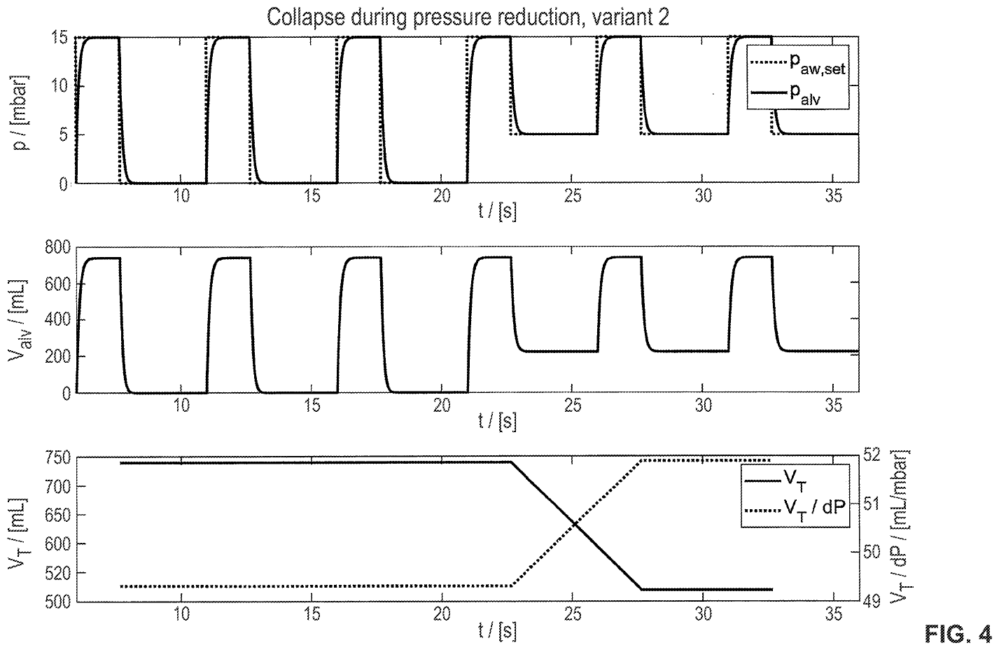

FIG. 4 schematically shows in a time diagram a response of collapsed lungs to a second ventilation pressure reduction. The first four breathing cycles take place with non-adapted ventilation parameters. The ventilation pressure is reduced by the fifth breathing cycle by raising PEEPₛₑₜ at constant P_{insp.set}. This brings about a reduction of the ventilation volume. The quotient of the ventilation volume (V₁) and the ventilation pressure (dP) (V_{T}/dP) increases in this case. The classification module 5 can recognize from this that a collapse of the lungs is present.

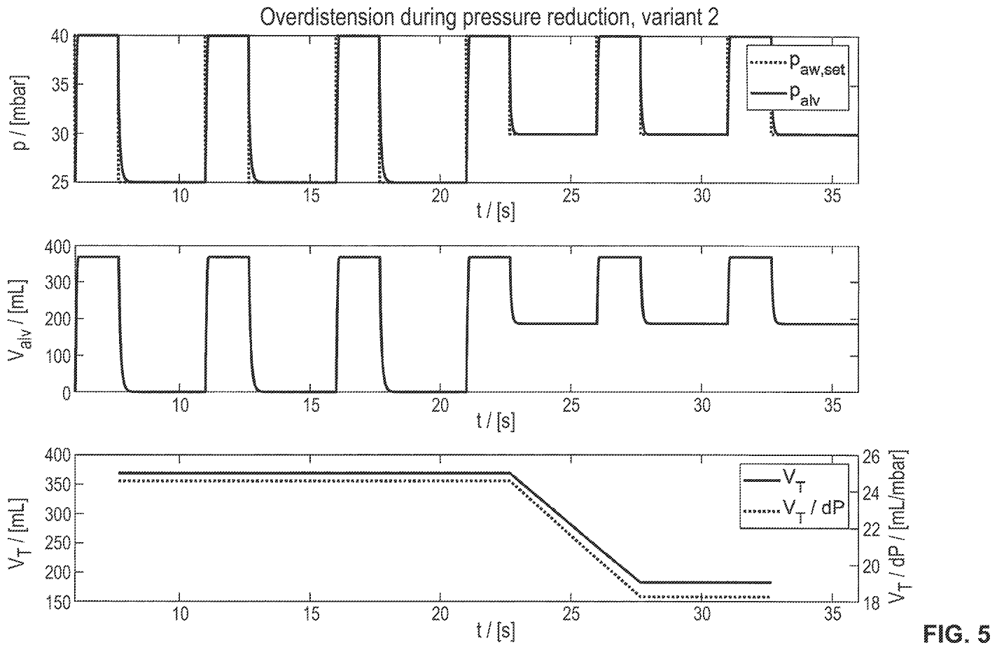

FIG. 5 schematically shows in a time diagram a response of overdistended lungs to the second ventilation pressure reduction. The first four breathing cycles take place with non-adapted ventilation parameters. The ventilation pressure is reduced by the fifth breathing cycle by raising PEEPₛₑₜ at constant P_{insp.set}. This brings about a reduction of the ventilation volume. The quotient of the ventilation volume (V₁) and ventilation pressure (dP) (V_{T}/dP) drops. The classification module 5 can recognize from this that an overdistension of the lungs is present.

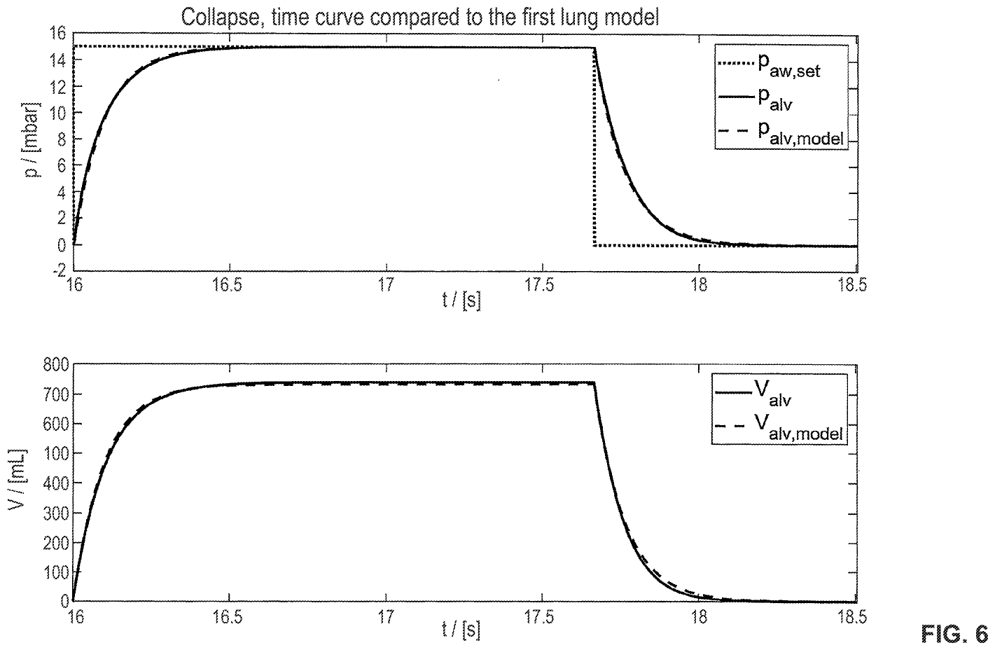

FIG. 6 schematically shows time diagrams of pressures and volumes of collapsed lungs (collapse) compared to a first linear lung model. The first linear lung model is estimated on the basis of the measured value curves of all breathing cycles. In the presence of an overdistension of the lungs, the compliance of the linear lung model is higher than the actual compliance at the time at which the plateau pressure is reached. Calculated ventilation volumes are thus higher than measured ventilation volumes. In addition, a rise time of the measured ventilation volume is shorter and a fall time is longer compared to the linear lung model, in which the rise time and the fall time are of equal length.

In the presence of a collapse of the lungs, the compliance of the linear lung model is lower than the actual compliance at the time at which the plateau pressure is reached. Calculated ventilation volumes are thus lower than measured ventilation volumes. Moreover, the rise time of the measured ventilation volume is longer and the fall time is shorter in the presence of a collapse of the lungs compared to the linear lung model.

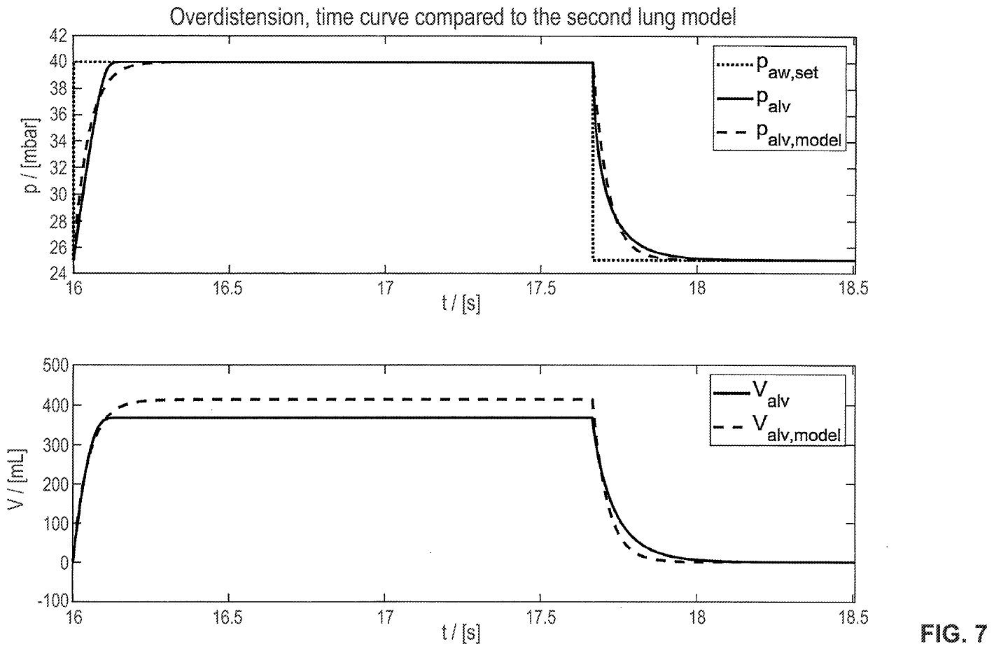

FIG. 7 schematically shows time diagrams of pressures and volumes of overdistended lungs (overdistension) compared to a second linear lung model. The second linear lung model is estimated separately for inhalation and exhalation only for the regions in which the value of the ventilation volume flow (q) exceeds a certain limit value. The linear lung model thus has an inspiratory lung model and an expiratory lung model.

The time constant, the rise time and the fall time of the inspiratory lung model are lower in the presence of an overdistension than those of the expiratory lung model.

The time constants, the rise time and the fall time of the inspiratory lung model are higher than those of the expiratory lung model in the presence of a collapse.

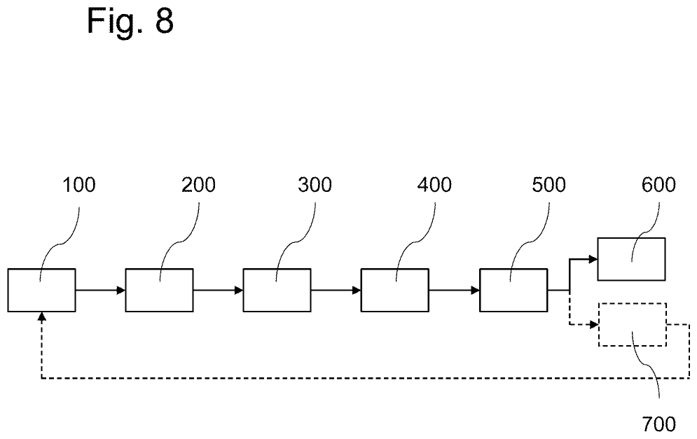

FIG. 8 schematically shows a preferred embodiment of the process according to the present invention in a flow chart. In a first process step 100, the breathing air flow for ventilating the patient is generated by means of the ventilation module 2 of the ventilator 1. The ventilation module 2 is controlled here by the control module 4. In a second process step 200, the first ventilation parameter and the second ventilation parameter are determined by means of the determination module 3 of the ventilator 1. The determination is preferably carried out continuously or repeatedly in order to guarantee a defined ventilation of the patient. In a third process step 300, the control device 4 of the ventilator 1 reduces the first ventilation parameter automatically over an analysis period comprising at least one breathing cycle. Either the P_{insp.set} is reduced here at constant PEEPₛₑₜ or PEEPₛₑₜ is raised at constant P_{insp.set}. In a fourth process step 400, the determination module 3 determines the change in the second ventilation parameter, which was brought about by the automatic reduction of the first ventilation parameter. In a fifth process step 500, the classification module 5 of the ventilator 1 classifies the pulmonary status of the lungs of the patient on the basis of the change in the second ventilation parameter, which change was brought about by the automatic reduction of the first ventilation parameter. Preferred classification categories are “overdistended,” “normal” and “collapsed.” In a sixth process step 600, the classified pulmonary status of the lungs of the patient and/or a recruitment maneuver suitable for improving the pulmonary status of the lungs are displayed by means of the display device 7 of the ventilator 1. As an alternative or in addition, a recruitment maneuver suitable for improving the pulmonary status of the lungs is carried out by means of the control device 4 in a seventh process step 700. The process is preferably carried out iteratively in order to attain successively a normal pulmonary status.

While specific embodiments of the invention have been shown and described in detail to illustrate the application of the principles of the invention, it will be understood that the invention may be embodied otherwise without departing from such principles.

1. A ventilator for ventilating the lungs of a patient with breathing air, the ventilator comprising:
a ventilation module configured to generate a breathing air flow;
a determination module configured to determine a first ventilation parameter as well as a second ventilation parameter of the ventilator, which said second ventilation parameter is different from the first ventilation parameter;
a control module configured to control the ventilator as a function of the determined first ventilation parameter and/or of the determined second ventilation parameter, wherein the control module is configured automatically to reduce the first ventilation parameter over an analysis period comprising at least one breathing cycle; and
a classification module configured to classify the pulmonary status of the lungs of the patient on the basis of a change in the second ventilation parameter, which was brought about by the automatic reduction of the first ventilation parameter.

2. A ventilator in accordance with claim 1, wherein the control module is configured to carry out a recruitment maneuver to improve the pulmonary status corresponding to a classification of the pulmonary status of the lungs of the patient, which was carried out by the classification module.

3. A ventilator in accordance with claim 1, wherein the classification module is configured to classify the pulmonary status of the lungs of the patient qualitatively as collapsed, overdistended or normal.

4. A ventilator in accordance with claim 1, wherein the classification module is configured to classify the pulmonary status of the lungs of the patient quantitatively.

5. A ventilator in accordance with claim 4, further comprising an alarm device configured to output an alarm when the quantitatively classified pulmonary status falls below a collapse limit value or exceeds an overdistension limit value.

6. A ventilator in accordance with claim 1, wherein the control module is configured to reduce a ventilation volume and/or a ventilation pressure automatically as a first ventilation parameter.

7. A ventilator in accordance with claim 1, wherein the control module is configured to reduce the first ventilation parameter stepwise over an analysis period comprising a plurality of breathing cycles.

8. A ventilator in accordance with claim 1, further comprising a display device, wherein the display device is configured to display the pulmonary status of the lungs of the patient and/or to display a recruitment maneuver recommended on the basis of the pulmonary status.

9. A ventilator , in accordance with claim 1, wherein the classification module is configured to estimate a linear lung model of the lungs of the patient on the basis of the first ventilation parameter and second ventilation parameter, which were determined prior to the automatic reduction of the first ventilation parameter, wherein the classification module is further configured to classify the pulmonary status of the lungs on the basis of the estimated lung model and on the basis of the second ventilation parameter determined after the automatic reduction of the first ventilation parameter.

10. A ventilator in accordance with claim 1, further comprising an EIT module for determining a pulmonary status of the lungs or at least a part of the lungs of the patient, wherein the classification module is configured to take into account a change in the distension and/or compliance of the lungs, which was brought about after the automatic reduction of the first ventilation parameter and was detected by the EIT module during the classification of the pulmonary status.

11. A ventilator in accordance with claim 1, wherein the control device is configured to reduce the first ventilation parameter automatically by between 20% and 60%.

12. A process for ventilating lungs of a patient with breathing air by means of a ventilator, the process comprising the steps of:
generating a breathing air flow by means of a ventilation module of the ventilator;
determining a first ventilation parameter and of a second ventilation parameter different from the first ventilation parameter by means of a determination module of the ventilator;
automatically reducing the first ventilation parameter over an analysis period comprising at least one breathing cycle by means of a control device of the ventilator;
determining a change in the second ventilation parameter, which change was brought about by the automatic reduction of the first ventilation parameter, by means of the determination module; and
classifying a pulmonary status of the lungs of the patient on the basis of the change in the second ventilation parameter, which was brought about by the automatic reduction of the first ventilation parameter, by means of a classification module of the ventilator.

13. A process in accordance with claim 12, wherein a breathing pressure is used as the first ventilation parameter and a ventilation volume is used as the second ventilation parameter.

14. A process in accordance with claim 12, wherein the classified pulmonary status of the lungs of the patient and/or a recruitment maneuver suitable for improving the pulmonary status of the lungs are displayed by means of a display device of the ventilator, and/or a recruitment maneuver suitable for improving the pulmonary status of the lungs is carried out by means of the control device.

15. A process in accordance with claim 12, wherein the control device is configured to carry out a recruitment maneuver to improve the pulmonary status corresponding to a classification of the pulmonary status of the lungs of the patient, which classification was carried out by the classification module.

16. A process in accordance with claim 12, wherein the classification module is configured to classify the pulmonary status of the lungs of the patient qualitatively as collapsed, overdistended or normal.

17. A process in accordance with claim 12, wherein the classification module is configured to classify the pulmonary status of the lungs of the patient quantitatively.

18. A process in accordance with claim 17, further comprising providing the ventilator with an alarm device configured to output an alarm when the quantitatively classified pulmonary status falls below a collapse limit value or exceeds an overdistension limit value.

19. A process in accordance with claim 12, wherein the control module is configured to reduce a ventilation volume and/or a ventilation pressure automatically as a first ventilation parameter.

20. A process in accordance with claim 12, wherein the control module is configured to reduce the first ventilation parameter stepwise over an analysis period comprising a plurality of breathing cycles.
